(19) United States
(12) Patent Application Publication
(10) Pub. No.: US 2006/0171936 A1
(43) Pub. Date: Aug. 3, 2006

(54) Cosmetic and/or dermatological composition for sensitive skin

(75) Inventors: Audrey Gueniche, Rueil Malmaison (FR); Lionel Breton, Versailles (FR); Olivier Ballevre, Shanghai (CN); Stephanie Blum-Sperisen, Mont-Pelerin (CH); Isabelle Bureau-Franz, Morges (CH); Jalil Benyacoub, Lausanne (CH)

Correspondence Address: OLIFF & BERRIDGE, PLC, P.O. BOX 19928, ALEXANDRIA, VA, 22320, US

(73) Assignees: L'OREAL, Paris (FR); NESTEC S.A., Vevey (CH)

(21) Appl. No.: 11/241,964

(22) Filed: Oct. 4, 2005

Related U.S. Application Data
(60) Provisional application No. 60/634,539, filed on Dec. 10, 2004.

(30) Foreign Application Priority Data
Oct. 4, 2004 (FR) 04 52258
Jun. 8, 2005 (EP) 05 012301.7

(51) Int. Cl. A61K 35/74

(52) U.S. Cl. 424/93.45

(57) ABSTRACT

The present invention relates to the use of an effective amount of at least one microorganism belonging to the species Lactobacillus paracasei or casei, a fraction thereof or a metabolite thereof, in combination with an effective amount of at least one microorganism belonging to the species Bifidobacterium longum or Bifidobacterium lactis, a fraction thereof or a metabolite thereof, for producing a dermatological composition intended for treating and/or preventing reactive sensitive skin that may or may not be associated with dryness of the skin.

### CROSS-REFERENCE TO RELATED APPLICATIONS

This application claims the benefit of French Application No. 04 52258 filed on Oct. 4, 2004, U.S. Provisional Application No. 60/634,539 filed on Dec. 10, 2004, and European Patent Application No. 05 012301.7 filed on Jun. 8, 2005, the entire disclosures of which are incorporated by reference herein.

### BACKGROUND

The present invention relates, in its main capacity, to the use of a combination of specific microorganisms for preparing a composition, in particular a cosmetic and/or dermatological composition, intended more particularly for the prevention and/or the treatment of skin described as sensitive and/or dry skin. It is also directed towards corresponding compositions.

In general, sensitive skin is defined by a particular reactivity of the skin. However, unlike skin described as allergic skin, this reactivity is not the result of an immunological process, i.e. does not occur only in a skin that has already been sensitized, in response to the presence of an allergen. Its mechanism is termed aspecific.

This skin reactivity is generally reflected by the manifestation of signs of discomfort in response to the individual coming into contact with a triggering element that may have various origins. This may be the application of a cosmetic product at the surface of the sensitive skin, food intake, or exposure to abrupt variations in temperature, to atmospheric pollution and/or to ultraviolet or infrared rays. Associated factors such as age and skin type also exist. Thus, sensitive skin is more common in the case of dry skin or oily skin than in the case of normal skin.

The appearance of these signs of discomfort, which appear within minutes following the individual coming into contact with the triggering element, is one of the essential characteristics of sensitive skin. It involves mainly dysesthetic sensations. The term “dysesthetic sensations” is intended to mean more or less painful sensations felt in an area of the skin, such as stinging, tingling, itching or pruritis, burning, hot sensations, discomfort, tautness, etc. These subjective signs exist most commonly in the absence of visible chemical signs such as redness and desquamations. It is today known that these skin irritation and intolerance reactions are in particular related to a release of neuropeptides by the nerve endings of the epidermis and of the dermis.

However, there is still no completely satisfactory solution available, at this time, for preventing and/or treating this type of skin described as sensitive, and this problem is more particularly exacerbated when this sensitive skin is associated with dry skin. Dry skin manifests itself essentially through a feeling of tautness and/or of tension, and it is often associated with a decrease in the level of moisturization of the skin and an impairment of the barrier function, measured through the imperceptible loss of water.

Document WO 02/28402 describes the fact that probiotic microorganisms can have a beneficial effect in the regulation of skin hypersensitivity reactions such as inflammatory and allergic reactions that result from an immunological process, as opposed to the reactivity of sensitive skin. In “Probiotics in the management of atopic eczema, Clinical and Experimental Allergy 2000”, Volume 30, pages 1604-1610, mention is also made of a study reporting the individual effect of some probiotics on infantile immune mechanisms such as, for example, atopic dermatitis.

### SUMMARY

Unexpectedly, the inventors have noted that a combination involved of at least two specific probiotic microorganisms is found to be most particularly effective, in particular in adults, for the treatment of sensitive skin, in particular associated with dry skin. The combination of microorganisms, considered according to the invention, advantageously shows a potential activity at the level of the skin barrier and a particular valency in maintaining the defence mechanisms, thus promoting maintenance of skin homeostasis and regulation of the skin's immune system.

According to a first of its aspects, the present invention relates to the use of an effective amount of at least one microorganism belonging to the species Lactobacillus paracasei or casei, a fraction thereof or a metabolite thereof, in combination with an effective amount of at least one microorganism belonging to the species Bifidobacterium longum or Bifidobacterium lactis, a fraction thereof or a metabolite thereof, for preparing a composition that is useful for preventing and/or treating sensitive and/or dry skin.

Consequently, according to another of its aspects, the present invention also relates to a cosmetic treatment method for preventing and/or treating sensitive and/or dry skin, comprising the administration, in particular oral or topical administration, of an effective amount of at least one microorganism belonging to the species Lactobacillus paracasei or casei, a fraction thereof or a metabolite thereof, in combination with an effective amount of at least one microorganism belonging to the species Bifidobacterium longum or Bifidobacterium lactis, a fraction thereof or a metabolite thereof.

According to a first variant, this combination is formulated in the form of a composition for oral absorption. It may in particular be a food supplement, or even a foodstuff.

According to a second variant, it is in the form of a cosmetic and/or dermatological composition according to the invention.

According to another of its aspects, the present invention also relates to a cosmetic and/or dermatological composition, that is in particular useful for preventing and/or treating sensitive and/or dry skin, comprising, in a physiologically acceptable carrier, an effective amount of at least one microorganism belonging to the species Lactobacillus paracasei ST11 NCC 2461 (CNCM I-2116), a fraction thereof or a metabolite thereof, in combination with an effective amount of at least one microorganism belonging to the species Bifidobacterium longum or Bifidobacterium lactis, a fraction thereof or a metabolite thereof.

According to another of its aspects, the present invention relates to a cosmetic and/or dermatological composition, that is in particular useful for preventing and/or treating sensitive and/or dry skin, comprising, in a physiologically acceptable carrier, an effective amount of at least one microorganism belonging to the species Lactobacillus paracasei or casei, a fraction thereof or a metabolite thereof, in combination with an effective amount of at least one microorganism belonging to the species Bifidobacterium longum or Bifidobacterium lactis, a fraction thereof or a metabolite thereof, and also comprising at least one divalent cation.

According to another of its aspects, the present invention relates to a cosmetic and/or dermatological composition, that is in particular useful for preventing and/or treating sensitive and/or dry skin, comprising, in a physiologically acceptable carrier, an effective amount of at least one microorganism belonging to the species Lactobacillus paracasei or casei, a fraction thereof or a metabolite thereof, in combination with an effective amount of at least one microorganism belonging to the species Bifidobacterium longum NCC 490 (CNCM I-2170) or Bifidobacterium lactis NCC 2818 (CNCM I-3446), a fraction thereof or a metabolite thereof.

According to another of its aspects, the present invention also relates to a composition for oral absorption, in particular of food supplement type, comprising in a physiologically acceptable carrier, an effective amount of at least one microorganism belonging to the species Lactobacillus paracasei ST11 NCC 2461 (CNCM I-2116), a fraction thereof or a metabolite thereof, in combination with an effective amount of at least one microorganism belonging to the species Bifidobacterium longum or Bifidobacterium lactis, a fraction thereof or a metabolite thereof.

According to another of its aspects, the present invention relates to a composition for oral absorption, in particular of food supplement type, comprising, in a physiologically acceptable carrier, an effective amount of at least one microorganism belonging to the species Lactobacillus paracasei or casei, a fraction thereof or a metabolite thereof, in combination with an effective amount of at least one microorganism belonging to the species Bifidobacterium longum or Bifidobacterium lactis, a fraction thereof or a metabolite thereof, and also comprising at least one divalent cation.

According to yet another of its aspects, the present invention relates to a composition for oral absorption, in particular of food supplement type, comprising, in a physiologically acceptable carrier, an effective amount of at least one microorganism belonging to the species Lactobacillus paracasei or casei, a fraction thereof or a metabolite thereof, in combination with an effective amount of at least one microorganism belonging to the species Bifidobacterium longum NCC 490 (CNCM I-2170) or Bifidobacterium lactis NCC 2818 (CNCM I-3446), a fraction thereof or a metabolite thereof.

### DETAILED DESCRIPTION OF EMBODIMENTS

As specified above, sensitive skin is different from allergic skin. Its reactivity is not the result of an immunological process and it is generally reflected only by dysesthetic sensations.

For obvious reasons, the lack of visible signs makes it difficult to diagnose sensitive skin. Most commonly, this diagnosis is based on the questioning of the patient. This symptomatology also has the advantage of making it possible to differentiate sensitive skin, that may or may not be associated with dry skin, from contact irritation or allergy for which there are, on the other hand, visible inflammatory signs.

Consequently, the development of “sensitive skin” products has required the availability of tools for evaluating the sensory reaction of the skin. The first tools were based, right from their design, on the essential characteristic of sensitive skin, namely the presence of signs of discomfort induced by a topical application. Thus, the lactic acid “stinging test” was the first test proposed. It is carried out by recording the stinging sensations reported by a volunteer after application of a 10% lactic acid solution to the wings of the nose. Individuals who report moderate or strong stinging sensations are called “stingers” and are considered to have sensitive skin. Because of this sensitivity of the skin to the topical application of a product, these individuals are then selected for testing “sensitive skin” products. More recently, in order to specifically activate the peripheral nerve endings involved in the discomfort and called nociceptors, recently identified as being involved in sensitive skin, new tests have been proposed that use precisely other inducers of discomfort such as capsaicin.

This second type of test, described in Application EP 1 374 913, also constitutes another particularly useful tool for diagnosing sensitive skin.

For the purpose of the present invention, sensitive skin covers irritable skin and intolerant skin.

Intolerant skin is skin that reacts to various factors, such as the application of cosmetic or dermatological products or soap, through sensations of heating, tautness, or tingling and/or redness. In general, these signs are associated with erythema and with hyper-seborrhoeaic or acneic skin, or even skin exhibiting rosacea, with or without sores.

Irritable skin is skin that reacts through pruritis, i.e. through itching or prickling, to various factors such as the environment, emotions, foods, the wind, rubbing, shaving, hard water with a high calcium concentration, temperature variations or wool.

In general, these two types of skin may be associated with dryness of the skin with or without sores or with skin that exhibits erythema.

As specified above, dryness of the skin is often associated with a decrease in the level of moisturization of the skin, evaluated by corneometry, and with an impairment of the barrier function, measured through the imperceptible loss of water.

Dry skin essentially manifests itself though a sensation of tautness and/or of tension. The said skin is also rough to the touch and appears to be covered with scales. When the skin is slightly dry, these scales are abundant but not very visible to the naked eye. When this condition worsens, there are increasingly fewer of these scales, but they are increasingly visible to the naked eye.

The cause of this dryness of the skin may be of the constitutional or acquired type.

In the case of acquired dry skin, the involvement of outside parameters such as exposure to chemical agents, to difficult climatic conditions or to sunlight, or alternatively certain therapeutic treatments (retinoids, for example) is determinant. Under these outside influences, the skin can then become momentarily and locally dry. This can involve any type of normal and even oily skin.

In the case of constitutionally dry skin, two categories can be distinguished: pathological skin and nonpathological skin.

Pathological constitutional dry skin is essentially represented by atopic dermatitis and ichthyoses. It is virtually independent of the outside conditions.

Atopic dermatitis is described as being associated with a deficiency in metabolism of the lipids of the stratum corneum, and in particular of the ceramides. This pathology presents itself in the form of more or less chronic xerosis concerning a large extent of the body, associated with inflammatory and pruriginous exacerbation in plaques.

Ichthyoses are pathologies characterized by a genetic deficiency that affects the keratinization process at various stages. They manifest themselves through considerable desquamation in plaques.

Nonpathological constitutional dry skin is dry skin for which the severity can depend on the outside factors already mentioned. Senile skin (characterized by a general decrease in metabolism with age), fragile skin (very sensitive to outside factors and often accompanied by erythema or rosacea) and common xerosis (of probable genetic origin and manifesting itself predominantly on the face, the limbs and the back of the hands) fall within this skin category.

The compositions and method according to the invention are thus found to be most particularly effective for preventing and/or treating sensitive and/or dry skin, and more particularly skin referred to as reactive, irritable and/or intolerant, acquired dry skin and/or constitutional dry skin.

The use and the compositions according to the invention employ at least one microorganism belonging to the species Lactobacillus paracasei or casei, in particular a microorganism belonging to the species Lactobacillus paracasei, and especially a microorganism belonging to the species Lactobacillus paracasei ST11 NCC 2461.

More particularly, the microorganism involved is Lactobacillus paracasei ST11 NCC 2461 deposited, according to the Treaty of Budapest, at the Pasteur Institute (28 rue du Docteur Roux—75024 Paris Cedex 15)—under the designation CNCM I-2116, a fraction thereof or a metabolite thereof.

As regards the microorganism belonging to the species Bifidobacterium longum, it may be more particularly be chosen from Bifidobacterium longum NCC 490 (CNCM I-2170). An other strain of Bifidobacterium longum is marketed under the name Bb 536 by MORINAGA.

As regards the microorganism belonging to the species Bifidobacterium lactis, it may more particularly be the species Bifidobacterium lactis NCC 2818 (CNCM I-3446).

According to a particular embodiment, the use and the compositions according to the invention employ at least the microorganism Lactobacillus paracasei ST11 NCC 2461 (CNCM I-2116), a fraction thereof or a metabolite thereof, and at least the microorganism Bifidobacterium longum NCC 490 (CNCM I-2170), a fraction thereof or a metabolite thereof.

According to another particular embodiment, the use and the compositions according to the invention employ the microorganism Lactobacillus paracasei ST11 NCC 2461 (CNCM I-2116), a fraction thereof or a metabolite thereof, and at least the microorganism Bifidobacterium lactis NCC 2818 (CNCM I-3446), a fraction thereof or a metabolite thereof.

For the purpose of the invention, the term “metabolite” denotes any substance derived from the metabolism of the microorganisms considered according to the invention and that is also effective for treating sensitive and/or dry skin.

For the purpose of the invention, the term “fraction” denotes more particularly a fragment of the said microorganism that is effective for treating dry and/or sensitive skin by analogy with the said whole microorganism.

Each of the corresponding microorganisms and/or metabolites and/or fractions can be formulated in a suitable carrier in a proportion of at least 10³ cfu/g, in particular in a proportion of 10⁵ to 10¹⁵ cfu/g and more particularly in a proportion of 10⁷ to 10¹² cfu/g.

According to a variant of the invention, they can be combined in a Lactobacillus paracasei or casei/Bifidobacterium longum or Lactobacillus paracasei or casei/Bifidobacterium lactis ratio, by cfu, ranging from 0.5 to 1.5, in particular from 0.7 to 1.2, and more particularly of the order of 1.

Advantageously, the use and the compositions according to the invention employ at least the said microorganisms in equivalent amounts and more particularly in a proportion of 10¹⁰cfu, respectively.

These microorganisms can be formulated in a powdered state, i.e. in a dry form, or in the form of suspensions or of solutions.

If necessary, these microorganisms can be formulated in an encapsulated form so as to significantly improve their survival time. In such a case, the presence of a capsule can in particular delay or prevent the degradation of the microorganism in the gastrointestinal tract.

These microorganisms can also be combined with at least one microorganism belonging to a different species, in particular a species of probiotic type and/or a fraction thereof and/or a metabolite thereof.

For the purpose of the present invention, the term “probiotic microorganism” is intended to mean a living mircoorganism that, when it is consumed in an appropriate amount, has a positive effect on the health of its host, “joint FAO/WHO Expert Consultation on Evaluation of Health and Nutritional Properties of Probiotic in Food Including Powder Milk with Live Lactic Acid Bacteria, 6 Oct. 2001”, and that can in particular improve the intestinal microbial balance.

These microorganisms that are suitable for the invention can be chosen in particular from ascomycetes such as Saccharomyces, Yarrowia, Kluyveromyces, Torulaspora, Schizosaccharomyces pombe, Debaromyces, Candida, Pichia, Aspergillus and Penicillium, bacteria of the genus Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus and Lactobacillus, and mixtures thereof.

As ascomycetes that are most particularly suitable for the present invention, mention may in particular be made of Yarrowia lipolitica and Kluyveromyces lactis, along with Saccharomyces cereviseae, Torulaspora, Schizosaccharamyces pombe, Candida and Pichia.

Specific examples of probiotic microorganisms are Bifidobacterium bifidum, Bifidobacterium infantis, Lactobacillus acidophilus, Lactobacillus alimentarius, Lactobacillus curvatus, Lactobacillus delbruckii subsp. Lactis, Lactobacillus gasseri, Lactobacillus johnsonii, Lactobacillus reuteri, Lactobacillus rhamnosus (Lactobacillus GG), Lactobacillus sake, Lactococcus lactis, Streptococcus thermophilus, Staphylococcus carnosus and Staphylococcus xylosus and mixtures thereof.

More particularly, they are probiotic microorganisms derived from the group of lactic acid bacteria, such as in particular Lactobacillus and/or Bifidobacterium. By way of illustration of these lactic acid bacteria, mention may more particularly be made of Lactobacillus johnsonii, Lactobacillus reuteri, Lactobacillus rhamnosus, Bifidobacterium bifidum, Bifidobacterium breve, Bifidobacterium animalis, Bifidobacterium lactis, Bifidobacterium longum, Bifidobacterium infantis, Bifidobacterium adolescentis or Bifidobacterium pseudocatenulatum and mixtures thereof.

A strain of Bifidobacterium lactis can be obtained from Hansen (Chr. Hansen A/S, 10-12 Boege Alle, P.O. Box 407, DK-2970 Hoersholm, Denmark) under the name Bb 12.

The species that are most particularly suitable are Lactobacillus johnsonii and Bifidobacterium adolescentis, respectively deposited, according to the Treaty of Budapest with the Pasteur Institute (28 rue du Docteur Roux, F-75024 Paris Cedex 15) under the following designations CNCM I-1225 and CNCM I-2168 and mixtures thereof.

In the case of a combination of at least one microorganism belonging to the species Lactobacillus paracasei or casei with at least one microorganism belonging to the species Bifidobacterium longum, the use or a composition according to the invention may also employ at least one microorganism belonging to the species Bifidobacterium lactis.

Similarly, in the case of a combination of at least one microorganism belonging to the species Lactobacillus paracasei or casei with at least one microorganism belonging to the species Bifidobacterium lactis, the use or a composition according to the invention may also employ at least one microorganism belonging to the species Bifidobacterium longum.

These microorganisms and/or fractions thereof and/or metabolites thereof may be formulated in a suitable carrier in an amount of at least 10³ cfu/g, in particular at doses ranging from 10⁵ to 10¹⁵ cfu/g, and more particularly of 10⁷ to 10¹² cfu/g of carrier.

The formulations disclosed above for the microorganisms belonging to the species constituting the combinations more particularly considered according to the invention, namely Lactobacillus paracasei or casei, Bifidobacterium longum and/or Bifidobacterium lactis can of course be considered for the abovementioned microorganisms.

In general, the compositions according to the invention, and in particular those intended to be administered orally, may comprise, for living microorganisms, from 10³ to 10¹⁵ cfu/g, in particular from 10⁵ to 10¹⁵ cfu/g and more particularly from 107 to 1012 cfu/g of microorganisms per gram of carrier or at calculated equivalent doses for inactive or dead microorganisms or for microorganism fractions or for metabolites produced. The compositions for topical application according to the invention generally comprise from 10³ to 10¹² cfu/g, in particular from 10⁵ to 10¹⁰ cfu/g and more particularly from 10⁷ to 10⁹ cfu/g of microorganisms, in particular probiotics.

When the composition comprises metabolites, the contents of metabolites in the compositions correspond substantially to the contents capable of being produced by 10³ to 10¹⁵ cfu, in particular 10⁵ to 10¹⁵ cfu, and more particularly 10⁷ to 10¹² cfu of living microorganisms per gram of carrier.

In the particular case of the compositions having to be administered orally, the concentration of each corresponding microorganism and/or fraction and/or metabolite can be adjusted so as to correspond to doses (expressed as microorganism equivalent) ranging from 5·10⁵ to 10¹³ cfu/day and in particular from 10⁸ to 10¹¹ cfu/day.

The microorganism(s) may be included in the composition according to the invention in a live, semi-active or inactivated, dead form.

It (they) may also be included in the form of fractions of cellular components or in the form of metabolites. The microorganism(s), metabolite(s) or fraction(s) may also be introduced in the form of a lyophilized powder, of a culture supernatant and/or, where appropriate, in a concentrated form.

In the particular case of topical compositions, it may be advantageous to use these microorganisms in inactivated, or even dead, form.

According to a variant of the invention, the use and the compositions according to the invention can also employ one or more divalent inorganic cation(s).

In the context of the present invention, the divalent inorganic cations can be used in various forms. The divalent inorganic cation can thus be in the form of an inorganic or organic, anhydrous or hydrated salt or of a chelated complex.

These salts may, for example, be carbonates, bicarbonates, sulphates, glycerophosphates, chlorides, nitrates, acetates, hydroxides, oxides, salts of a-hydroxy acids (citrates, tartrates, lactates, malates) or of fruit acids, or else salts of amino acids (aspartate, arginate, fumarate) or salts of fatty acids (palmitate, oleate, caseinate, behenate).

According to a particular embodiment, the divalent inorganic cation is chosen from manganese, copper and/or zinc.

According to another particular embodiment, the divalent inorganic cation is an alkaline earth metal. As alkaline earth metals that can be used in the invention, mention may be made of barium, calcium, magnesium, strontium and/or beryllium.

Advantageously, the divalent inorganic cation, and in particular alkaline earth metal, is used in the present invention in the form of a salt. In particular, the salt may be chosen from calcium nitrate, strontium nitrate, magnesium gluconate, calcium lactate, strontium gluconate, magnesium lactate, calcium chloride, strontium chloride, magnesium chloride, calcium carbonate, strontium sulphate, magnesium sulphate, calcium glycerophosphate, calcium citrate, magnesium citrate, strontium acetate, magnesium acetate and mixtures thereof.

According to a particularly advantageous embodiment, at least one divalent inorganic cation chosen from strontium, calcium and/or magnesium citrate, chloride, gluconate, sulphate, lactate and/or acetate salts, and mixtures thereof, is used.

The divalent inorganic cation may also be used in the form of a chelated complex, in particular chelated with crystalline or ionized proteins.

The divalent inorganic cation may also be in a specific form that is stored by a microorganism, for example of the yeast type, like selenium yeast.

Thus, the cations can be introduced as they are into the compositions according to the invention, or by means of a compound or mixture of compound(s) known to contain a high concentration of at least one of these cations. For example, as source of metal salts, use may be made of an extract of plants or yeasts rich in cations. Similarly, calcium may for example be introduced via a milk extract.

The divalent inorganic cation content used in the compositions according to the invention depends, of course, on the form of the cation under consideration, and can be determined by means of simple routine experiments. These daily doses can in particular range from 100 μg to 5 g, more particularly from 1 mg to 2 g, or even from 10 mg to 1.3 g.

In the compositions intended for oral administration according to the invention, the divalent inorganic cation concentration can be adjusted so as to correspond to doses ranging from 1 to 3000 mg/day and in particular from 10 to 2000 mg/day.

The compositions according to the invention are generally administered topically or orally.

The compositions according to the invention may be in any of the pharmaceutical forms normally used according to the route used.

The carrier may be of various nature according to the type of composition under consideration.

Food or pharmaceutical carriers that are especially suitable include milk, yoghourt, cheese, fermented milks, milk-based fermented products, ice-creams, fermented cereal-based products, milk-based powders, formulas for children and infants, foods for animals, in particular pets, tablets or lozenges, liquid bacterial suspensions, oral supplements in dry form and oral supplements in liquid form.

In particular, the composition according to the invention may be a food composition for human consumption. It may in particular be completely nutritional foods, drinks, mineral waters, soups, dietetic supplements and replacement foods, nutritional bars, confectionery, milk-based or fermented milk-based products, yoghourts, milk-based powders, enteral nutrition products, compositions for children and/or infants, cereal-based products or fermented cereal-based products, ice-creams, chocolate, coffee, “culinary” products such as mayonnaise, tomato puree or salad dressings. The composition according to the invention may also be intended for animals.

As regards more particularly the cosmetic products, they may be aqueous, aqueous-alcoholic or oily solutions, dispersions of the solution type or dispersions of the lotion or serum type, emulsions having a liquid or semi-liquid consistency of the milk type, suspensions or emulsions, of the cream type, an aqueous or anhydrous gel, microemulsions, microcapsules, microparticles, or vesicular dispersions of ionic and/or non-ionic type.

For ingestion, many embodiments of oral compositions and in particular of food supplements are possible. They are formulated by means of the usual methods for producing dragées, gelatine capsules, gels, emulsions, tablets, capsules or solutions. In particular, the active agent(s) according to the invention can be incorporated into any other forms of food supplements or of enriched foods, for example, food bars, or compacted or non-compacted powders. The powders can be diluted with water, in a fizzy drink, dairy products or soya-derived products, or can be incorporated into food bars.

The active agents according to the invention can be formulated with the usual excipients and constituents for such oral compositions or food supplements, i.e. in particular fatty and/or aqueous constituents, humectifying agents, thickeners, texturing, flavouring and/or coating agents, antioxidants, preserving agents and dyes that are usual in the food sector.

The formulating agents and excipients for oral compositions and, in particular for food supplements, are known in this sector and are not, here, the subject of a detailed description.

Of course, the oral compositions according to the invention may contain several other active agents.

As active agents that can be used, mention may be made of vitamins B3, B5, B6, B8, C, E, or PP, carotenoids, curcuminoids and niacin.

In particular, use may be made of an antioxidant complex comprising vitamins C and E, and at least one carotenoid, in particular a carotenoid chosen from β-carotene, lycopene, astaxanthine, zeaxanthine and lutein, flavonoids such as catechins, hesperidin, proanthocyanidins and anthocyanins.

The composition advantageously comprises at least one prebiotic or a mixture of prebiotics. More particularly, these prebiotics can be chosen from oligosaccharides, produced from glucose, galactose, xylose, maltose, sucrose, lactose, starch, xylan, hemicellulose, inulin, gums, of the acacia type for example, or a mixture thereof. More particularly, the oligosaccharide comprises at least one fructooligosaccharide. More paticularly, this prebiotic may comprise a mixture of fructooligosaccharide and of inulin.

The cosmetic and/or dermatological compositions more particularly relating to a topical application can in particular be in the form of aqueous, aqueous-alcoholic or oily solutions, of dispersions of the solution type or dispersions of the lotion or serum type, of emulsions that have a liquid or semi-liquid consistency of the milk type, obtained by dispersion of a fatty phase in an aqueous phase (O/W) or vice versa (W/O), or of suspensions or emulsions that have a soft, semi-solid or solid consistency, of the cream, aqueous gel or anhydrous gel type, or else of microemulsions, of microcapsules, of microparticles, or of vesicular dispersions of ionic and/or non-ionic type.

These compositions are prepared according to the usual methods.

These compositions can in particular constitute cleansing, protective, treatment or care creams for the face, for the hands, for the feet, for the major anatomical folds or for the body (for example, day creams, night creams, makeup-removing creams, foundation creams, sun creams), makeup products such as fluid foundations, makeup-removing milks, protective or care milks for the body, aftersun milks, skincare lotions, gels or foams, such as cleansing or disinfecting lotions, sun lotions, artificial tanning lotions, bath compositions, deodorant compositions containing a bactericidal agent, aftershave gels or lotions, depilatory creams, or compositions for preventing insect bites.

The compositions according to the invention may also consist of solid preparations constituting soaps or cleansing bars.

They may also be used for the hair, in the form of aqueous, alcoholic or aqueous-alcoholic solutions or in the form of creams, gels, emulsions, or mousses or else in the form of an aerosol composition also containing a pressurized propellant.

When the composition of the invention is an emulsion, the proportion of the fatty phase can range from 5 to 80% by weight, and preferably from 5 to 50% by weight, relative to the total weight of the composition. The oils, the emulsifiers and the coemulsifiers used in the composition in the form of an emulsion are chosen from those conventionally used in the cosmetics and/or dermatological fields. The emulsifier and the coemulsifier may be present, in the composition, in a proportion ranging from 0.3 to 30% by weight, and preferably from 0.5 to 20% by weight, relative to the total weight of the composition.

When the composition of the invention is an oily solution or gel, the fatty phase can represent more than 90% of the total weight of the composition.

In a known manner, the cosmetic and/or dermatological composition of the invention can also contain adjuvants that are normal in the cosmetics, pharmaceutical and/or dermatological fields, such as hydrophilic or lipophilic gelling agents, hydrophilic or lipophilic active agents, preserving agents, antioxidants, solvents, fragrances, fillers, screening agents, bactericides, odour absorbers and dyestuffs. The amounts of these various adjuvants are those conventionally used in the field under consideration and are, for example, from 0.01 to 20% of the total weight of the composition. Depending on their nature, these adjuvants can be introduced into the fatty phase and/or into the aqueous phase.

As fats that can be used in the invention, mention may be made of mineral oils such as, for example, hydrogenated polyisobutene and liquid petroleum jelly, plant oils, such as, for example, a liquid fraction of shea butter, sunflower oil and apricot kernel oil, animal oils such as for example perhydrosqualene, synthetic oils, in particular Purcellin oil, isopropyl myristate, ethylhexyl palmitate, and fluoro oils such as for example, perfluoropolyethers. Use may also be made of fatty alcohols, fatty acids such as, for example, stearic acid and such as, for example, waxes, in particular paraffin wax, carnauba wax and beeswax. Use may also be made of silicone compounds such as silicone oils and, for example, cyclomethicone and dimethicone, and waxes, resins and gums that contain silicone. These compounds may or may not be functionalized.

As emulsifiers that can be used in the invention, mention may, for example, be made of glycerol stearate, polysorbate 60, the mixture of cetyl stearyl alcohol/oxyethylenated cetyl stearyl alcohol containing 33 mol of ethylene oxide, sold under the name Sinnowax AO® by the company Henkel, the mixture of PEG-6/PEG-32/glycol stearate sold under the name Tefose®63 by the company Gattefosse, PPG-3 myristyl ether, silicone emulsifiers such as cetyl dimethicone copolyol and sorbitan monostearate or tristearate, PEG-40 stearate, and oxyethylenated (20 EO) sorbitan monostearate.

As solvents that can be used in the invention, mention may be made of lower alcohols, in particular ethanol and isopropanol, and propylene glycol.

As hydrophilic gelling agents, mention may be made of carboxylic polymers such as carbomer, acrylic copolymers such as acrylate/alkyl acrylate copolymers, polyacrylamides and in particular the mixture of polyacrylamide, C13-14-Isoparaffin and Laureth-7, sold under the name Sepigel 305® by the company Seppic, polysaccharides for instance cellulose derivatives such as hydroxyalkylcelluloses and in particular hydroxypropylcellulose and hydroxyethylcellulose, natural gums such as guar, carob et xanthan gums, and clays.

As lipophic gelling agents, mention may be made of modified clays such as bentones, metal salts of fatty acids such as aluminium stearates and hydrophobic silica, or else ethylcellulose and polyethylene.

As hydrophilic activate agents, use may be made of proteins or protein hydrolysates, amino acids, polyols, in particular C₂ to C₁₀ polyols such as glycerol, sorbitol, butylene glycol and polyethylene glycol, urea, allantoin, sugars and sugar derivatives, water-soluble vitamins, starch, bacterial or plant extracts such as those of aloe vera.

As lipophilic active agents, use may be made of retinol (vitamin A) and its derivatives, tocopherol (vitamin E) et its derivatives, ceramides and essential oils.

The active agents according to the invention can also be combined with active agents intended in particular for the prevention and/or the treatment of skin conditions.

In addition, the composition of the invention can advantageously contain a spring and/or mineral water, in particular chosen from Vittel water, water from the Vichy basin and Roche Posay water.

The cosmetic treatment method of the invention can be carried out in particular by applying the cosmetic and/or dermatological compositions as defined above according to the normal technique for using these compositions, for example: application of creams, gels, sera, lotions, makeup-removing milks or aftersun compositions to the skin or to dry hair, application of a hair lotion to wet hair, application of shampoos, or else application of dentifrice to the gums.

The cosmetic methods, according to the invention can be carried out by a topical administration or by oral administration, daily, for example, of the combination according to the invention, which may, for example, be formulated in the form of gelatine capsules, gels, lotions, dragées, emulsions, tablets, capsules or oral ampoules, in an appropriate amount and number according to their form, so that the active agents are administered at a rate of 5·10⁵ to 10¹³ cfu per day, in particular 10⁶ to 10¹¹ cfu per day, in terms of microorganisms or at equivalent doses in terms of partially inactivated or dead microorganisms or in terms of microorganism fractions or of metabolites produced.

According to another embodiment, the administration is repeated until the divalent inorganic cation is administered at doses of the order of 1 to 3000 mg per day, and in particular of 10 to 2000 mg per day.

The method according to the invention can comprise a single administration. According to another embodiment, the administration is repeated, for example 2 to 3 times daily, over a day or more, and generally over a prolonged period of at least 4 weeks, or even 4 to 15 weeks, with, where appropriate, one or more periods of interruption.

In the description and in the following examples, unless otherwise indicated, the percentages are percentages by weight and the ranges of values written as “between . . . and . . . ” include the upper and lower limits specified. The ingredients are mixed, before they are formulated, in the order and under conditions that are readily determined by those skilled in the art.

The examples hereinafter are given by way of nonlimiting illustration of the field of the invention.

### EXAMPLES OF COMPOSITIONS FOR ORAL ADMINISTRATION

### Example 1

### Powder Stick

| | | |
|---|---|---|
| Active principle | | |
| Lactobacillus paracasei ST11 NCC 2461 (CNCM I-2116) | 10¹⁰ | cfu |
| Bifidobacterium longum NCC 490 (CNCM I-2170) | 10¹⁰ | cfu |
| Calcium citrate | 50 | mg |
| Excipient | | |
| Xanthan gum | 0.8 | mg |
| Sodium benzoate | 0.2 | mg |
| Maltodextrin | qs 30 | g |

One stick per day can be taken.

### Example 2

### Powder Stick

| | | |
|---|---|---|
| Active principle | | |
| Lactobacillus paracasei ST11 NCC 2461 (CNCM I-2116) | 10¹⁰ | cfu |
| Bifidobacterium longum NCC 490 (CNCM I-2170) | 10¹⁰ | cfu |
| Magnesium citrate | 50 | mg |
| Excipient | | |
| Xanthan gum | 0.8 | mg |
| Sodium benzoate | 0.2 | mg |
| Maltodextrin | qs 30 | g |

One stick per day can be taken.

### Example 3

### Capsule

| Active principle | mg/capsule |
|---|---|
| Lactobacillus paracasei ST11 NCC 2461 (CNCM I-2116) | 10⁸ cfu |
| Bifidobacterium longum NCC 490 (CNCM I-2170) | 10⁸ cfu |
| Magnesium gluconate | 150 |
| Vitamin C | 60 |
| Magnesium stearate | 0.02 |

One to three of the capsules can be taken per day.

### Example 4

### Formulation of Dragée Type

| | mg/dragée |
|---|---|
| Active materials | |
| Magnesium gluconate | 50 |
| Lactobacillus paracasei ST11 NCC 2461 (CNCM I-2116) | 5 · 10⁸ cfu |
| Bifidobacterium longum NCC 490 (CNCM I-2170) | 5 · 10⁸ cfu |
| Calcium citrate | 200 |
| Excipient of the core of the dragée | |
| Microcrystalline cellulose | 70 |
| Encompress ™ | 60 |
| Magnesium stearate | 3 |
| Anhydrous colloidal silica | 1 |
| Coating agent | |
| Shellac | 5 |
| Talc | 61 |
| Sucrose | 250 |
| Polyvidone | 6 |
| Titanium dioxide | 0.3 |
| Colorant | 5 |

This type of dragée can be taken 1 to 3 times per day.

### Example 5

### Formulation of Dragée Type

| | mg/dragée |
|---|---|
| Active materials | |
| Magnesium lactate | 50 |
| Bifidobacterium longum NCC 490 (CNCM I-2170) | 10⁹ cfu |
| Lactobacillus paracasei ST11 NCC 2461 (CNCM I-2116) | 10⁹ cfu |
| Calcium citrate | 200 |
| Excipient of the core of the dragée | |
| Microcrystalline cellulose | 70 |
| Encompress ™ | 60 |
| Magnesium stearate | 3 |
| Anhydrous colloidal silica | 1 |
| Coating agent | |
| Shellac | 5 |
| Talc | 61 |
| Sucrose | 250 |
| Polyvinylidone | 6 |
| Titanium dioxide | 0.3 |
| Colorant | 5 |

This type of dragée can be taken 1 to 3 times per day.

### Example 6

### Powder Stick

| | | |
|---|---|---|
| Active principle | | |
| Lactobacillus paracasei ST11 NCC 2461 (CNCM I-2116) | 10¹⁰ | cfu |
| Bifidobacterium lactis NCC 2818 (CNCM I-3446) | 10¹⁰ | cfu |
| Calcium citrate | 50 | mg |
| Excipient | | |
| Xanthan gum | 0.8 | mg |
| Sodium benzoate | 0.2 | mg |
| Maltodextrin | qs 30 | g |

One stick per day can be taken.

### Example 7

### Powder Stick

| | | |
|---|---|---|
| Active principle | | |
| Lactobacillus paracasei ST11 NCC 2461 (CNCM I-2116) | 10¹⁰ | cfu |
| Bifidobacterium lactis NCC 2818 (CNCM I-3446) | 10¹⁰ | cfu |
| Magnesium citrate | 50 | mg |
| Excipient | | |
| Xanthan gum | 0.8 | mg |
| Sodium benzoate | 0.2 | mg |
| Maltodextrin | qs 30 | g |

One stick per day can be taken.

### Example 8

### Capsule

| Active principle | mg/capsule |
|---|---|
| Lactobacillus paracasei ST11 NCC 2461 (CNCM I-2116) | 10⁸ cfu |
| Bifidobacterium lactis NCC 2818 (CNCM I-3446) | 10⁸ cfu |
| Magnesium gluconate | 150 |
| Vitamin C | 60 |
| Magnesium stearate | 0.02 |

One to three of these capsules can be taken per day.

### Example 9

### Formulation of Dragée type

| | mg/dragée |
|---|---|
| Active materials | |
| Magnesium gluconate | 50 |
| Lactobacillus paracasei ST11 NCC 2461 (CNCM I-2116) | 5 · 10⁸ cfu |
| Bifidobacterium lactis NCC 2818 (CNCM I-3446) | 5 · 10⁸ cfu |
| Calcium citrate | 200 |
| Excipient of the core of the dragée | |
| Microcrystalline cellulose | 70 |
| Encompress ™ | 60 |
| Magnesium stearate | 3 |
| Anhydrous colloidal silica | 1 |
| Coating agent | |
| Shellac | 5 |
| Talc | 61 |
| Sucrose | 250 |
| Polyvidone | 6 |
| Titanium dioxide | 0.3 |
| Colorant | 5 |

This type of dragée can be taken 1 to 3 times per day.

### Example 10

### Formulation of Dragée Type

| | mg/dragée |
|---|---|
| Active materials | |
| Magnesium lactate | 50 |
| Bifidobacterium lactis NCC 2818 (CNCM I-3446) | 10⁹ cfu |
| Lactobacillus paracasei ST11 NCC 2461 (CNCM I-2116) | 10⁹ cfu |
| Calcium citrate | 200 |
| Excipient of the core of the dragée | |
| Microcrystalline cellulose | 70 |
| Encompress ™ | 60 |
| Magnesium stearate | 3 |
| Anhydrous colloidal silica | 1 |
| Coating agent | |
| Shellac | 5 |
| Talc | 61 |
| Sucrose | 250 |
| Polyvinylidone | 6 |
| Titanium dioxide | 0.3 |
| Colorant | 5 |

This type of dragée can be taken one to three times per day.

### EXAMPLES OF COMPOSITION FOR TOPICAL ADMINISTRATION

### Example 11

### Sensitive Skin Facial Lotion

| | |
|---|---|
| Lactobacillus paracasei ST11 NCC 2461 (CNCM I-2116) | 5.00 |
| powder | |
| Bifidobacterium longum NCC 490 (CNCM I-2170) powder | 5.00 |
| Magnesium gluconate | 3.00 |
| Calcium lactate | 2.00 |
| Antioxidant | 0.05 |
| Isopropanol | 40.0 |
| Preserving agent | 0.30 |
| Water | qs 100% |

### Example 12

### Dry and Sensitive Skin Facial Care Milk

| | |
|---|---|
| Magnesium chloride | 3.00 |
| Calcium ascorbate | 3.00 |
| Lactobacillus paracasei ST11 NCC 2461 (CNCM I-2116) | 5.00 |
| powd2er | |
| Bifidobacterium longum NCC 490 (CNCM I-2170) powder | 5.00 |
| Glyceryl stearate | 1.00 |
| Cetyl stearyl alcohol/oxyethylenated cetyl stearyl alcohol | 3.00 |
| containing 30 mol of EO (Sinnowax AO ® sold by the | |
| company Henkel) | |
| Cetyl alcohol | 1.00 |
| Dimethicone (DC 200 Fluid ® sold by the company | 1.00 |
| Dow Corning) | |
| Liquid petroleum jelly | 6.00 |
| Isopropyl myristate (Estol IPM 1514 ® sold by Unichema) | 3.00 |
| Antioxidant | 0.05 |
| Glycerol | 20.00 |
| Preserving agent | 0.30 |
| Water | qs 100 |

### Example 13

### Sensitive Skin Facial Care Gel

| | |
|---|---|
| Strontium nitrate | 4.00 |
| Lactobacillus paracasei ST11 NCC 2461 (CNCM I-2116) | 5.00 |
| powder | |
| Bifidobacterium longum NCC 490 (CNCM I-2170) powder | 5.00 |
| Hydroxypropylcellulose (Klucel H ® sold by the company | 1.00 |
| Hercules) | |
| Vitamin E | 2.50 |
| Antioxidant | 0.05 |
| Isopropanol | 40.00 |
| Preserving agent | 0.30 |
| Water | qs 100% |

### Example 14

### Dry and Sensitive Skin Facial Care Milk

| | |
|---|---|
| Magnesium ascorbate | 3.00 |
| Blackcurrant seed oil | 4.00 |
| Borage oil | 4.00 |
| Lactobacillus paracasei ST11 NCC 2461 (CNCM I-2116) | 5.00 |
| powder | |
| Bifidobacterium longum NCC 490 (CNCM I-2170) powder | 5.00 |
| Glyceryl stearate | 1.00 |
| Cetyl stearyl alcohol/oxyethylenated cetyl stearyl alcohol | 3.00 |
| containing 3 mol of EO (Sinnowax AO ® sold by the | |
| company Henkel) | |
| Cetyl alcohol | 1.00 |
| Dimethicone (DC 200 Fluid ® sold by the company | 1.00 |
| Dow Corning) | |
| Liquid petroleum jelly | 6.00 |
| Isopropyl myristate (Estol IPM 1514 ® sold by the | 3.00 |
| company Unichema) | |
| Glycerol | 20.00 |
| Preserving agent | 0.30 |
| Water | qs 100 |

### Example 15

### Sensitive Skin Facial Lotion

| | |
|---|---|
| Lactobacillus paracasei ST11 NCC 2461 (CNCM I-2116) | 5.00 |
| powder | |
| Bifidobacterium lactis NCC 2818 (CNCM I-3446) powder | 5.00 |
| Magnesium gluconate | 3.00 |
| Calcium lactate | 2.00 |
| Antioxidant | 0.05 |
| Isopropanol | 40.0 |
| Preserving agent | 0.30 |
| Water | qs 100% |

### Example 16

### Dry and Sensitive Skin Facial Care Milk

| | |
|---|---|
| Magnesium chloride | 3.00 |
| Calcium ascorbate | 3.00 |
| Lactobacillus paracasei ST11 NCC 2461 (CNCM I-2116) | 5.00 |
| powder | |
| Bifidobacterium lactis NCC 2818 (CNCM I-3446) powder | 5.00 |
| Glyceryl stearate | 1.00 |
| Cetyl stearyl alcohol/oxyethylenated cetyl stearyl alcohol | 3.00 |
| containing 30 mol EO (Sinnowax AO ® sold by the | |
| company Henkel) | |
| Cetyl alcohol | 1.00 |
| Dimethicone (DC 200 Fluid ® sold by the company | 1.00 |
| Dow Corning) | |
| Liquid petroleum jelly | 6.00 |
| Isopropyl myristate (Estol IPM 1514 ® sold by Unichema) | 3.00 |
| Antioxidant | 0.05 |
| Glycerol | 20.00 |
| Preserving agent | 0.30 |
| Water | qs 100 |

### Example 17

### Sensitive Skin Facial Care Gel

| | |
|---|---|
| Strontium nitrate | 4.00 |
| Lactobacillus paracasei ST11 NCC 2461 (CNCM I-2116) | 5.00 |
| powder | |
| Bifidobacterium lactis NCC 2818 (CNCM I-3446) powder | 5.00 |
| Hydroxypropylcellulose (Klucel H ® sold by the company | 1.00 |
| Hercules) | |
| Vitamin E | 2.50 |
| Antioxidant | 0.05 |
| Isopropanol | 40.00 |
| Preserving agent | 0.30 |
| Water | qs 100% |

### Example 18

### Dry and Sensitive Skin Facial Care Milk

| | |
|---|---|
| Magnesium ascorbate | 3.00 |
| Blackcurrant seed oil | 4.00 |
| Borage oil | 4.00 |
| Lactobacillus paracasei ST11 NCC 2461 (CNCM I-2116) | 5.00 |
| powder | |
| Bifidobacterium lactis NCC 2818 (CNCM I-3446) powder | 5.00 |
| Glyceryl stearate | 1.00 |
| Cetyl stearyl alcohol/oxyethylenated cetyl stearyl alcohol | 3.00 |
| containing 3 mol EO (Sinnowax AO ® sold by the | |
| company Henkel) | |
| Cetyl alcohol | 1.00 |
| Dimethicone (DC 200 Fluid ® sold by the company | 1.00 |
| Dow Corning) | |
| Liquid petroleum jelly | 6.00 |
| Isopropyl myristate (Estol IPM 1514 ® sold by the | 3.00 |
| company Unichema) | |
| Glycerol | 20.00 |
| Preserving agent | 0.30 |
| Water | qs 100 |

### Example 19

### Effectiveness Study

An oral composition based on a probiotic microorganism (B) was tested for its effectiveness with respect to skin dryness and sensitivity, with regard to a placebo composition (A). They have the following composition:

A: Maltodextrin

B: 1×10¹⁰ cfu Lactobacillus paracasei ST11 NCC 2461 (CNCM I-2116)+1×10¹⁰ cfu Bifidobacterium lactis NCC 2818 (CNCM I-3446).

The treatment consists of the daily and oral administration of a single treatment unit for a period of eight weeks.

This study was carried out on 66 adult female individuals whose age is between 18 and 50, and who were identified following a clinical evaluation (clinical score for dryness of legs and roughness of the face) and a self-evaluation by means of a questionnaire (validated sensitive skin questionnaire) as individuals with dry and sensitive skin. These 66 individuals were divided up into 2 parallel groups of 33 individuals, with one of the groups receiving the product tested and one of the groups receiving the placebo.

The effect of the supplement tested is assessed by comparison with the control “placebo” formulation. The results obtained are given in Table I below.

TABLE I

| | |
|---|---|
| % variation between D1 and D57 | Food supplement based on |
| is significant versus placebo¹ | probiotics according to |
| | the invention |
| | (B) |
| Clinical score: decrease compared | |
| with D1 | |
| Roughness of the face | −79% (p = 0.06) |
| Dryness of the legs | −60% (p = 0.02) |
| Self-evaluation: decrease compared | |
| with D1 | |
| Dryness of the legs | −28% (p = 0.2) |
| Bioanalysis: Skin sensitivity | −60% (p = 0.02) |
| Moisturization factor: increase compared | |
| with D1 | |
| Urea | +28% (p = 0.6) |
| Sodium lactate | Maintenance of level |
| | (p = 0.15) although decrease |
| | of 60% with the placebo |

¹Analysis of contrasts between D1 and D57 between the treated group and the placebo group.

The oral composition in accordance with the invention, of the example, was tested in terms of skin sensitivity in the individuals considered for the study (evaluation of skin sensitivity by means of a lactic acid test or stinging test).

A reduction in skin sensitivity of approximately −60% (p=0.02) was thus observed between D1 and D57 in the treated individuals.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-36. (canceled)

37. A method of producing a composition intended for treating and/or preventing reactive sensitive skin that may be associated with dryness of the skin, comprising combining:
an effective amount of at least one first microorganism belonging to the species Lactobacillus paracasei or casei, a fraction thereof or a metabolite thereof; and
an effective amount of at least one second microorganism belonging to the species Bifidobacterium longum or Bifidobacterium lactis, a fraction thereof or a metabolite thereof.

38. The method of claim 37, wherein the first microorganism belonging to the species Lactobacillus paracasei or casei is Lactobacillus paracasei ST11 NCC 2461 (CNCM I-2116).

39. The method of claim 37, wherein the second microorganism belonging to the species Bifidobacterium lactis is Bifidobacterium lactis NCC 2818 (CNCM I-3446).

40. The method of claim 37, wherein the second microorganism belonging to the species Bifidobacterium longum is Bifidobacterium longum NCC 490 (CNCM I-2170).

41. The method of claim 37, wherein:
the first microorganism belonging to the species Lactobacillus paracasei or casei is Lactobacillus paracasei ST11 NCC 2461 (CNCM I-2116); and
the second microorganism belonging to the species Bifidobacterium longum is Bifidobacterium longum NCC 490 (CNCM I-2170).

42. The method of claim 37, wherein:
the first microorganism belonging to the species Lactobacillus paracasei or casei is Lactobacillus paracasei ST11 NCC 2461 (CNCM I-2116); and
the second microorganism belonging to the species Bifidobacterium lactis is Bifidobacterium lactis NCC 2818 (CNCM I-3446).

43. The method of claim 37, wherein the first and second microorganisms, fractions thereof and/or metabolites thereof are formulated in a physiologically acceptable carrier in an amount of at least 10³ cfu/g of carrier.

44. The method of claim 37, wherein a ratio of an amount of the first microorganisms, fractions thereof and/or metabolites thereof in cfu to an amount of the second microorganisms, fractions thereof and/or metabolites thereof in cfu is from 0.5 to 1.5.

45. The method of claim 37, comprising further combining a third microorganism, a fraction thereof or a metabolite thereof.

46. The method of claim 45, wherein the third microorganism is of probiotic type.

47. The method of claim 45, wherein the third microorganism comprises at least one member selected from the group consisting of: Saccharomyces, Yarrowia, Kluyveromyces, Torulaspora, Schizosaccharomyces pombe, Debaromyces, Candida, Pichia, Aspergillus, Penicillium, Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus and Lactobacillus.

48. The method of claim 45, wherein the third microorganism comprises at least one member selected from the group consisting of: Saccharomyces cereviseae, Yarrowia lipolitica, Kluyveromyces lactis, Torulaspora, Schizosaccharomyces pombe, Candida, Pichia, Bifidobacterium bifidum, Bifidobacterium breve, Bifidobacterium animalis, Bifidobacterium lactis, Bifidobacterium longum, Bifidobacterium infantis, Bifidobacterium adolescentis, Bifidobacterium pseudocatenulatum, Lactobacillus acidophilus, Lactobacillus alimentarius, Lactobacillus curvatus, Lactobacillus delbruckii subsp. Lactis, Lactobacillus gasseri, Lactobacillus johnsonii, Lactobacillus reuteri, Lactobacillus rhamnosus (Lactobacillus GG), Lactobacillus sake, Lactococcus lactis, Streptococcus thermophilus, Staphylococccus carnosus and Staphylococcus xylosus.

49. The method of claim 45, wherein the third microorganism is derived from lactic acid bacteria.

50. The method of claim 45, wherein the third microorganism comprises at least one member selected from the group consisting of: Lactobacillus johnsonii (CNCM I-1225) and Bifidobacterium adolescentis (CNCM I-2168).

51. The method of claim 37, comprising further combining at least one divalent inorganic cation.

52. The method of claim 51, wherein the divalent inorganic cation is an alkaline earth metal.

53. The method of claim 52, wherein the alkaline earth metal is at least one member selected from the group consisting of barium, calcium, magnesium, strontium and beryllium.

54. The method of claim 52, wherein the divalent inorganic cation is in the form a salt selected from the group consisting of carbonates, bicarbonates, sulphates, glycerophosphates, chlorides, nitrates, acetates, hydroxides, oxides, citrates, tartrates, lactates, malates, salts of fruit acid, aspartates, arginates, fumarates, palmitates, oleates, caseinates and behenates.

55. A cosmetic treatment method for preventing and/or treating sensitive skin that may be associated with dry skin, comprising orally or topically administering at least an effective amount a composition comprising:
at least one first microorganism belonging to the species Lactobacillus paracasei or casei, a fraction thereof or a metabolite thereof; and
at least one second microorganism belonging to the species Bifidobacterium longum or Bifidobacterium lactis, a fraction thereof or a metabolite thereof.

56. The method of claim 55, wherein the first microorganism belonging to the species Lactobacillus paracasei or casei is Lactobacillus paracasei ST11 NCC 2461 (CNCM I-2116).

57. The method of claim 55, wherein the second microorganism belonging to the species Bifidobacterium lactis is Bifidobacterium lactis NCC 2818 (CNCM I-3446).

58. The method of claim 55, wherein the second microorganism belonging to the species Bifidobacterium longum is Bifidobacterium longum NCC 490 (CNCM I-2170).

59. The method of claim 55, wherein:
the first microorganism belonging to the species Lactobacillus paracasei or casei is Lactobacillus paracasei ST11 NCC 2461 (CNCM I-2116); and
the second microorganism belonging to the species Bifidobacterium longum is Bifidobacterium longum NCC 490 (CNCM I-2170).

60. The method of claim 55, wherein:
the first microorganism belonging to the species Lactobacillus paracasei or casei is Lactobacillus paracasei ST11 NCC 2461 (CNCM I-2116); and
the second microorganism belonging to the species Bifidobacterium lactis is Bifidobacterium lactis NCC 2818 (CNCM I-3446).

61. The method of claim 55, wherein the first and second microorganisms, fractions thereof and/or metabolites thereof are formulated in a physiologically acceptable carrier in an amount of at least 10³ cfu/g of carrier.

62. The method of claim 55, wherein a ratio of an amount of the first microorganisms, fractions thereof and/or metabolites thereof in cfu to an amount of the second microorganisms, fractions thereof and/or metabolites thereof in cfu is from 0.5 to 1.5.

63. The method of claim 55, wherein orally or topically administering at least an effective amount of the composition comprises administering the first and second microorganisms, fractions and/or metabolites at a rate of from 5×10⁵ to 10¹³ cfu per day.

64. A cosmetic and/or dermatological composition, comprising:
an effective amount of at least one first microorganism belonging to the species Lactobacillus paracasei or casei, a fraction thereof or a metabolite thereof;
an effective amount of at least one second microorganism belonging to the species Bifidobacterium longum or Bifidobacterium lactis, a fraction thereof or a metabolite thereof; and
a physiologically acceptable carrier.

65. The composition of claim 64, wherein the first microorganism belonging to the species Lactobacillus paracasei or casei is Lactobacillus paracasei ST11 NCC 2461 (CNCM I-2116).

66. The composition of claim 64, further comprising at least one divalent inorganic cation.

67. The composition of claim 64, wherein the second microorganism belonging to the species Bifidobacterium longum or Bifidobacterium lactis is Bifidobacterium longum NCC 490 (CNCM I-2170) or Bifidobacterium lactis NCC 2818 (CNCM I-3446).
